(19) United States
(12) Patent Application Publication
(10) Pub. No.: US 2003/0109496 A1
(43) Pub. Date: Jun. 12, 2003

(54) Fluid insecticidal formulations for treatment of parasitic insect larvae by dermal application

(76) Inventors: Kirkor Sirinyan, Bergisch Gladbach (DE); Dorothee Stanneck, Solingen (DE)

Correspondence Address: BAYER CORPORATION, PATENT DEPARTMENT, 100 BAYER ROAD, PITTSBURGH, PA, 15205, US

(21) Appl. No.: 10/149,603

(22) PCT Filed: Dec. 5, 2000
(86) PCT No.: PCT/EP00/12194

(30) Foreign Application Priority Data
Dec. 16, 1999 (DE) 199607753

(51) Int. Cl. A61K 31/695; A01N 55/00

(52) U.S. Cl. 514/63

(57) ABSTRACT

The present invention relates to the use of polysiloxanes containing at least one quaternary ammonium group as formulation auxiliary in formulations of larvicidal and/or ovicidal active compounds and to compositions containing

a) a larvicidal and/or ovicidal active compound and

b) a polysiloxane derivative with at least one quaternary ammonium group per molecule,

and, if appropriate, further auxiliaries and carriers.

The present invention relates to the use of polysiloxanes containing at least one quaternary ammonium group for the preparation of new, storage-stable, skin-compatible, dermally applicable liquid formulations of larvicidal and/or ovicidal active compounds for controlling parasitic insect larvae on animals.

In the preparation of active compound formulations in the fields of pharmacology and veterinary medicine, there is frequently, in particular in the case of aqueous formulations, the problem that the active compounds lack solubility, and, in this context, that the finished formulations lack storage stability.

When larvicidal and/or ovicidal active compounds, some of which are sparingly soluble in water, are applied in the form of dermally applicable liquid formulations, it is therefore necessary to prepare homogeneous solutions based on organic solvents and insecticidal active compounds. To do this, the active compounds are usually dissolved in organic solvents such as isopropanol, 2-butoxy-ethyl acetate, ethylene glycol diacetate, and, if appropriate, the solutions are mixed with further additives. The preparation of such formulations is described in U.S. Pat. No. 4,874,753, EP-A 137 627 and GB-A 2 135 886. The disadvantages of said systems when larvicidal and/or ovicidal active compounds are used are that frequently they result in irritations of the skin and furthermore, in the customary spot-on primary packing materials, still have a relatively poor long-term stability and thus do not meet the basic requirements of the official pharmacopoeias regarding long-term stability behaviour.

It is desirable to replace these formulations with formulations which have outstanding compatibility with the skin, are toxicologically acceptable and are furthermore distinguished by their very good long-term action of several weeks and their long-term stability of several years in all climatic zones in the conventional spot-on primary packaging materials.

To remedy this disadvantage, for example of the known formulations, patent specifications AU-A 627 847, EP-A 413 610 propose that these active compounds be dissolved in high-boiling solvents such as monopropylene glycol which additionally also contain natural, skin-compatible oils such as pine oil, sunflower oil or soya oil. Furthermore, it can be seen from patent specification WO 91/13545 that highly effective, skin-compatible liquid formulations can be prepared by dissolving these active compounds in amounts of >50% in aliphatic solvents such as 2-(2-butoxy-ethoxy)ethanol or 2-(2-methoxy-ethoxy)ethanol. The disadvantage of these formulations is that they require the use of substantial amounts of active compound and, furthermore, result in irritations of the skin in sensitive animal breeds. To achieve an acceptable biological action by using small amounts of active compound, patent specification U.S. Pat. No. 5,466,458 proposes the use of emulsions based on these active compounds together with long-chain aliphatic amines or alcohols such as hexadecan-1-ol, 1-octadecylamine. The use of the long-chain amines has the disadvantage that they eventually break down these active compounds. In most cases, the formulations based on long-chain alcohols do not have sufficient long-term action.

The object of the present invention is therefore to provide a skin-compatible, environmentally and user-friendly formulation based on larvicidal and/or ovicidal active compounds which is outstandingly stable in conventional spot-on primary packaging materials as specified in the drug product regulations and is highly effective against parasitic insect larvae.

EP-A 0 017 121, 0 017 122, 0 282 720, 0 294 642, 0 166 122 and 0 164 668 disclose a large number of polysiloxanes with terminal quaternary amino groups and their use as conditioners in hair shampoos and haircare products.

Surprisingly, the objects set out above are now achieved by the compositions according to the invention using the abovementioned polysiloxanes. The result is clear solutions or emulsions with high storage stability.

Moreover, the use according to the invention of these polysiloxanes surprisingly results in an improved compatibility and an activity-enhancing, synergistic effect.

Accordingly, the present invention relates to the use of polysiloxanes containing at least one quaternary ammonium group as formulation auxiliary in formulations of larvicidal and/or ovicidal active compounds.

Subject-matter of the present application are furthermore new compositions containing

a) a larvicidal and/or ovicidal active compound and

b) a polysiloxane derivative containing at least one quaternary ammonium group per molecule,

and, if appropriate, further auxiliaries and carriers.

In particular, the compositions according to the invention are outstandingly suitable for the preparation of spot-on and pour-on compositions for use in the control of parasites on animals.

Larvicidal and/or ovicidal active compounds based on juvenile hormones and fluorobenzoylureas may be employed for the preparation of the liquid formulations according to the invention, juvenile hormones which act against flea larvae being especially preferred. Juvenile hormones which act against parasitic insect larvae are known (see, in this context, GB-A 2 140 010, German Offenlegungsschrift 37 00 881, German Offenlegungsschrift 38 25 172). Likewise, the larvicidal and/or ovicidal action of fluorobenzoylureas is known and can be seen from the literature (see, for example, EP-A 343 110, German Offenlegungsschrift 38 27 133, EP-A 230 400, EP-A 255 803).

The following active compounds are especially suitable:

Juvenile hormones and juvenile hormone analogues such as:

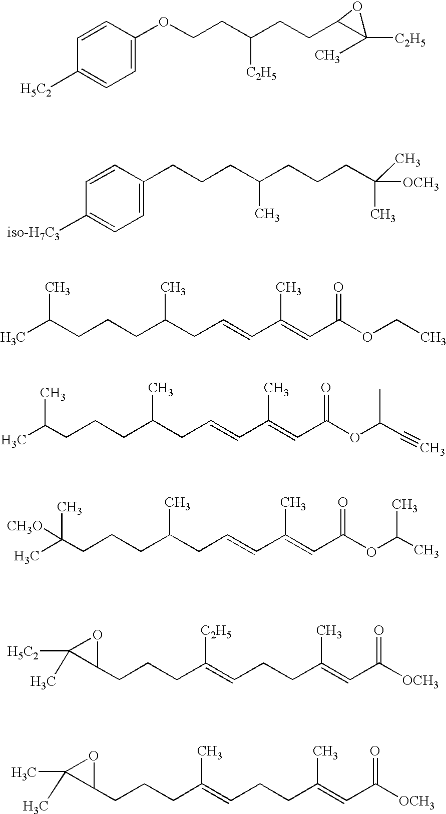

Substituted diaryl ethers such as:

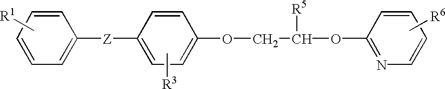

| R¹ | R³ | R⁵ | R⁶ | Z |
|---|---|---|---|---|
| H | H | CH₃ | H | O |
| H | H | CH₃ | 2-Cl | O |
| 5-F | H | CH₃ | H | O |
| H | H | CF₃ | H | O |
| H | H | C₂H₅ | H | O |
| H | H | H | H | O |
| H | H | CH₃ | H | CH₂ |
| H | H | CH₃ | H | C(CH₃)₂ |

Benzoylureas such as:

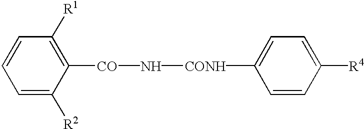

| R¹ | R² | R⁴ |
|---|---|---|
| H | Cl | CF₃ |
| Cl | Cl | CF₃ |
| F | F | CF₃ |
| H | F | CF₃ |
| H | Cl | SCF₃ |
| F | F | SCF₃ |
| H | F | SCF₃ |
| H | Cl | OCF₃ |
| F | F | OCF₃ |
| H | F | OCF₃ |
| F | F | 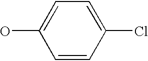 |
| F | F | 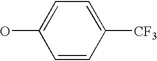 |
| F | F | 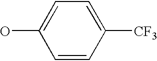 |

The use of pyriproxyfen:

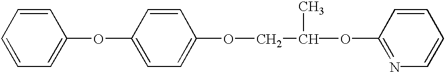

may be particularly emphasized.

The amounts of active compound may be varied within a wide range of 0.1-15%. Amounts ranging from 0.1-7.5% are preferred. Amounts of 0.1-5% are especially preferably employed. Amounts ranging from 0.2-2.0% are very especially preferably employed for preparing the new formulations according to the invention. Percentages are by weight.

Naturally, further active compounds may be employed as components for combination in the compositions according to the invention.

Active compounds which may be used in combinations are preferably the insecticides employed in the field of controlling ectoparasitic insects such as nicotinyl insecticides and, in particular, chloronicotinyl insecticides, or n-phenyl-pyrazoles, carbamates, phosphoric and phosphonic esters, growth inhibitors, or mixtures of these active compounds with each other and their mixtures with synergists. Synergists for the purposes of the present application are understood as meaning compounds which themselves do not have the desired activity, but, when used as a component in mixtures, lead to an increased activity of the actual active compounds.

Chloronicotinyl insecticides which may be mentioned are compounds of the formulae (I), (II) and (III):

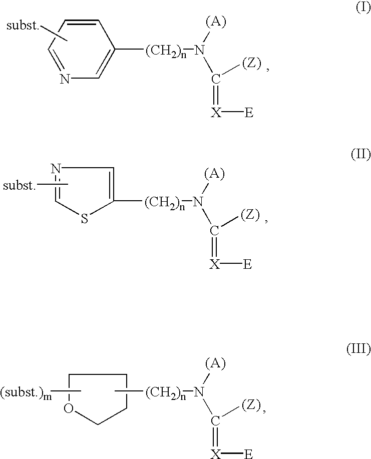

where

n represents 1 or 2,

m represents 0, 1 or 2,

subst. represents one of the abovementioned substituents, in particular halogen, very especially chlorine, and

A, Z, X and E have the abovementioned meanings.

The following compounds may be mentioned individually:

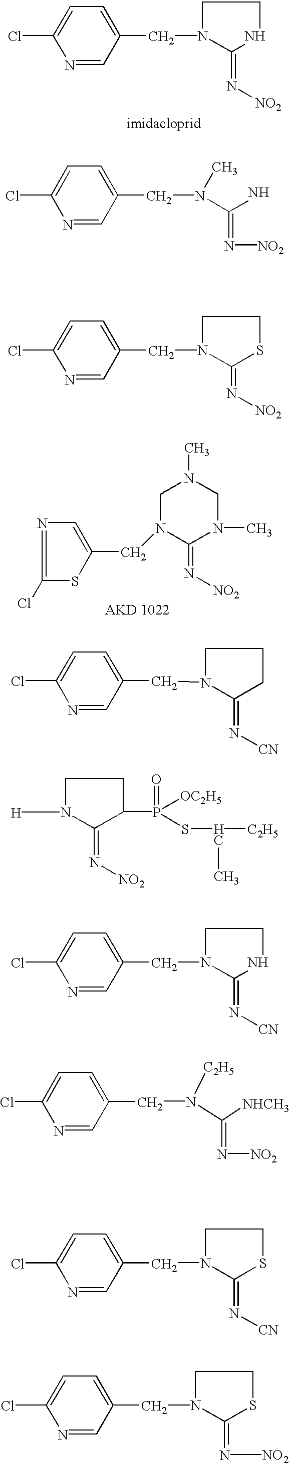

The following compounds may be emphasized in particular:

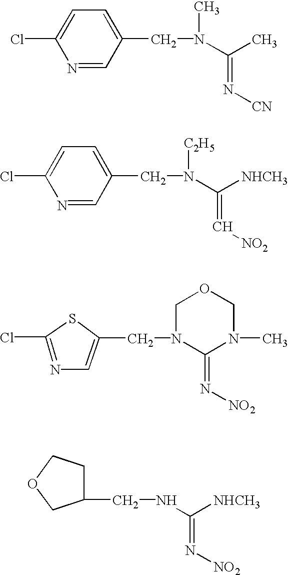

Furthermore, the following compounds may be emphasized in particular:

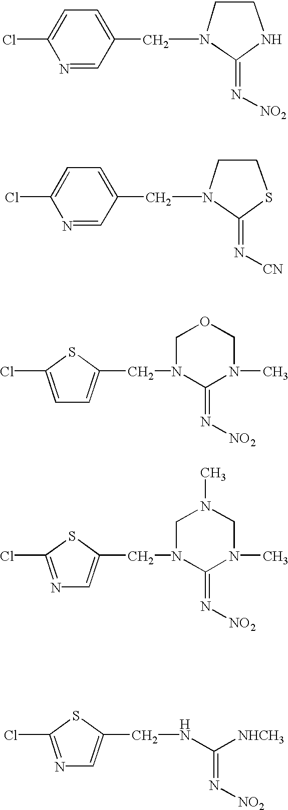

Carbamates which may be mentioned are substituted phenyl- and naphthyl-carbamates

Examples of phenylpyrazoles which may be mentioned are the following compounds:

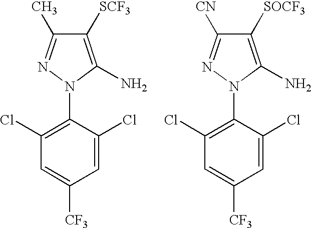

The following may be mentioned as being preferred:

2-osobutylphenyl N-methylcarbamate,

4-dimethylamino-3-methyl-phenyl N-methylcarbamate,

2-isopropoxy-phenyl N-methylcarbamate,

1-naphthyl N-methylcarbamate,

m-tolyl N-methylcarbamate,

3,4-xylyl N-methylcarbamate,

3,5-xylyl N-methylcarbamate,

2-[1,3-dioxolan-2-yl]-phenyl N-methylcarbamate.

Phosphoric esters which may be mentioned as being preferred are the compounds with the common names phoxim, fenitrothion, dichlorvos, trichlorfon and malathion.

Juvenile hormones and juvenile hormone analogues such as

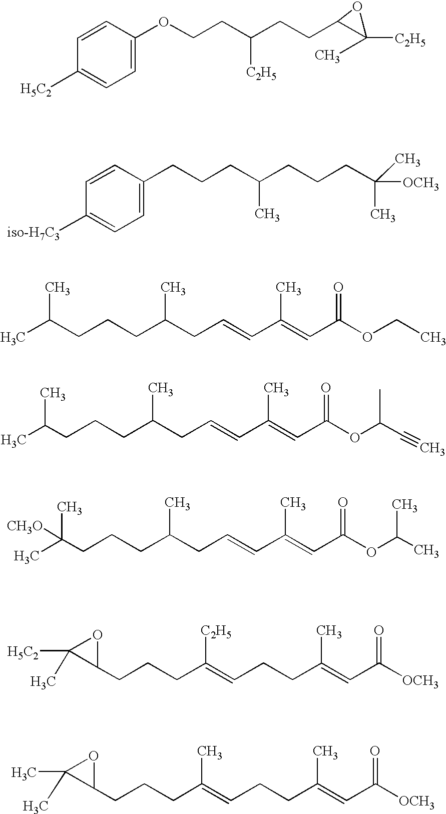

Substituted diarylethers such as:

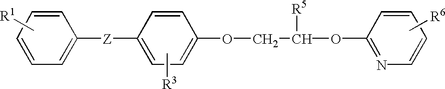

| R¹ | R³ | R⁵ | R⁶ | Z |
|---|---|---|---|---|
| H | H | CH₃ | H | O |
| H | H | CH₃ | 2-Cl | O |
| 5-F | H | CH₃ | H | O |
| H | H | CF₃ | H | O |
| H | H | C₂H₅ | H | O |
| H | H | H | H | O |
| H | H | CH₃ | H | CH₂ |
| H | H | CH₃ | H | C(CH₃)₂ |

Benzoylureas such as:

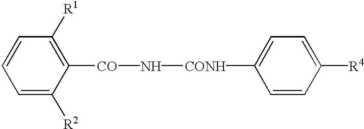

| R¹ | R² | R⁴ |
|---|---|---|
| H | Cl | CF₃ |
| Cl | Cl | CF₃ |
| F | F | CF₃ |
| H | F | CF₃ |
| H | Cl | SCF₃ |
| F | F | SCF₃ |
| H | F | SCF₃ |
| H | Cl | OCF₃ |
| F | F | OCF₃ |
| H | F | OCF₃ |
| F | F | 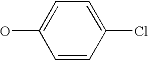 |
| F | F | 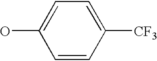 |
| F | F | 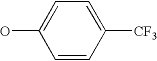 |

Triazines such as:

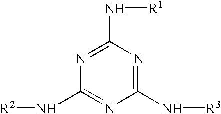

| R¹ | R² | R³ |
|---|---|---|
| cyclopropyl | H | H |
| cyclopropyl | H | CH₃ |
| cyclopropyl | H | C₂H₅ |
| cyclopropyl | H | C₃H₇-n |
| cyclopropyl | H | C₄H₉-n |
| cyclopropyl | H | C₅H₁₁-n |
| cyclopropyl | H | C₆H₁₃-n |
| cyclopropyl | H | C₇H₁₅-n |
| cyclopropyl | H | C₈H₁₇-n |
| cyclopropyl | H | C₁₂—H₂₅-n |
| cyclopropyl | H | CH₂—C₄H₉-n |
| cyclopropyl | H | CH₂CH(CH₃)C₂H₅ |
| cyclopropyl | H | CH₂CH═CH₂ |
| cyclopropyl | Cl | C₂H₅ |
| cyclopropyl | Cl | C₆H₁₃-n |
| cyclopropyl | Cl | C₈H₁₇-n |
| cyclopropyl | Cl | C₁₂H₂₅-n |
| cyclopropyl | H | cyclopropyl |
| cyclopropyl | H | COCH₃ |
| cyclopropyl | H | COCH₃ HCl |
| cyclopropyl | H | COC₂H₅ HCl |
| cyclopropyl | H | COC₂H₅ |
| cyclopropyl | H | COC₃H₇-n |
| cyclopropyl | H | COC₃H₇-i |
| cyclopropyl | H | COC₄H₉-t HCl |
| cyclopropyl | H | COC₄H₉-n |
| cyclopropyl | H | COC₆H₁₃-n |
| cyclopropyl | H | COC₁₁—H₂₃-n |
| cyclopropyl | COCH₃ | COC₂H₅ |
| cyclopropyl | COC₃H₇-n | COC₆H₁₃-n |
| cyclopropyl | COCH₃ | COC₃H₇-n |
| cyclopropyl | COC₂H₅ | COC₃H₇-n |
| cyclopropyl | H | CO-cyclopropyl |
| cyclopropyl | CO-cyclopropyl | CO-cyclopropyl |
| cyclopropyl | COCH₃ | COCH₃ |
| isopropyl | H | H |
| isopropyl | H | COCH₃ |
| isopropyl | H | COC₃H₇-n |
| cyclopropyl | H | CONHCH₃ |
| cyclopropyl | H | CONHC₃H₇-i |
| cyclopropyl | CONHCH₃ | CONHCH₃ |
| cyclopropyl | H | SCNHCH₃ |
| cyclopropyl | H | CONHCH₂CH═CH₂ |
| cyclopropyl | CONHCH₂CH═CH₂ | CONHCH₂CH═CH₂ |
| cyclopropyl | CSNHCH₃ | CSNHCH₃ |

The amounts of the active compounds which may be used in combinations may be varied within a wide range of 0.1 to 12.5%, the amounts ranging from 0.1 to 10.0% being especially preferred and the amounts ranging from 0.5 to 7.5% being very especially preferred. Percentages are by weight.

Synergists for these compounds which may be mentioned as being preferred are piperonyl butoxide and sesame seed oil. These synergists are described, for example, in patent specification EP-413 610.

The selected formulation auxiliaries based on polydimethyl siloxanes with cationic quaternary amine groups of the formula

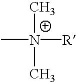

R′=various organic radicals

are known polymeric or oligomeric compounds. Polysiloxanes which may be mentioned by way of example but not by way of limitation are those described in EP-A 0 017 121, p. 2, 1. 11 to p. 3, 1. 3, EP-A 0 017 122, p. 2, 1. 11 to p. 3, 1. 13, EP-A 0 166 122, p. 4, 1. 31 to p. 7, end, EP-A 0 294 642, p. 5, 1. 10 to p. 8, 1. 51, EP-A 282 720, p. 6, 1. 10 to p. 14, 1. 54 and those in EP-A 0 164 668 on p. 4, 1. 31 to p. 8, 1. 3.

To prepare the compositions according to the invention, it is possible to employ polysiloxanes which have monoquatemary amine groups, but also those which have polyquaternary amine groups. Naturally, these polydimethylsiloxanes may have further functional groups such as carboxyl, amine, hydroxyl, carboxylate groups. The functional groups which are very especially preferred are hydroxyl and carboxyl groups. Their viscosity may be varied within a wide range of 200 to 17,500 mm²s⁻¹ (at 25° C.), measured as specified by DIN 53 019 as a 50% aqueous solution, those with a viscosity ranging from 250 to 10,000 mm²s⁻¹ (at 25° C.) being especially preferable and those with a viscosity ranging from 250 to 1,350 mm²s⁻¹ (at 25° C.) being very especially preferable.

Very especially preferred are polysiloxanes of the formula (V)

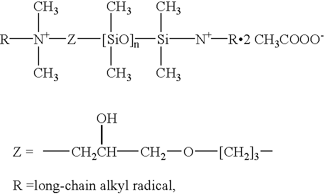

which are sold by Goldschmidt under the trade name ABIL®.

The amounts of polydimethylsiloxane employed may be varied within a wide range of 0.1-15%, amounts ranging from 0.1-5% being preferable. Amounts between 0.1 and 2.5% are especially preferred. Amounts ranging from 0.25-2.5% are very especially preferably employed for producing the new formulations according to the invention. Percentages are by weight.

Solvents according to the invention which are employed are aliphatic polyethers such as diethylene glycol monomethyl ether, dipropylene glycol monopropyl ether, cyclic carbonates such as propylene carbonate, ethylene carbonate, aliphatic and aromatic alcohols such as ethanol, isopropanol, acetates such as benzyl acetate, benzyl benzoate, or mixtures of these with each other.

Solvents which are especially preferred are aliphatic polyethers, in particular diethylene glycol monomethyl ether, dipropylene glycol monopropyl ether or diethylene glycol monomethyl ether. Dipropylene glycol monopropyl ether are very especially preferably employed for preparing the new liquid formulations. It is also possible to employ mixtures of the above-mentioned solvents.

In the formulation according to the invention, the organic solvent amounts to 2.5 to 99.8% by weight, preferably to 60 to 99.0% by weight, especially preferably to 60 to 93.7% by weight, very especially preferably to 65-90% by weight, and in particular to 75-83% by weight.

The amount of water in the new formulations may be varied within a wide range of 9-95% by weight, 0-30% by weight being preferred, 7.5-30% by weight especially preferred, 7.5-25% by weight very especially preferable, and 12.5-17.5% by weight particularly preferable.

In addition, the formulations according to the invention may contain customary auxiliaries such as antioxidants or flavour-masking agents.

Stabilizers and antioxidants which may be mentioned are sulphites or metabisulphites such as potassium metabisulphite, organic acids such as citric acid, ascorbic acid; phenols, butylhydroxytoluene, butylhydroxyanisole, or tocopherol, the organic acids citric acid and malic acid being preferable. Stabilizers which are very especially preferred are citric acid and butylhydroxytoluene. They may be varied within a wide range of 0.05 to 2.5% by weight, amounts ranging from 0.075-0.15% by weight being especially preferred.

Examples of flavour-masking agents are mixtures of organic fatty acid esters. They preferably amount to 0.1 to 2% by weight in the formulations according to the invention.

Surprisingly, the liquid formulations according to the invention are distinguished by an outstanding storage stability of several years in all climatic zones, and by skin compatibility, compatibility with the user and the environmental. Surprisingly, they are outstandingly suitable for packaging and marketing in single-dose polypropylene polymer tubes with a wall thickness of 300-500 μm and a filling volume of 1.0 to 4.0 ml, which are usually liable to storage problems.

Moreover, the liquid formulations according to the invention exhibit a synergistic, i.e. activity-enhancing, effect which could not have been expected, for example when using pyriproxyfen as active compound.

The compositions according to the invention are environmentally compatible and, owing to the fact that their toxicity is very low, user-friendly.

The compositions according to the invention have a favourable toxicity to warm-blooded species and are suitable for controlling parasitic insects found in animal keeping and animal breeding in domestic animals and livestock, but also in zoo animals, laboratory animals, experimental animals and pets. They are active against all or individual developmental stages of the pests and to resistant and normally sensitive species of pests.

The pests include:

from the order of the Anoplura, for example, Haematopinus spp., Linognathus spp., Solenopotes spp., Pediculus spp., Pthirus spp.;

from the order of the Mallophaga, for example, Trimenopon spp., Menopon spp., Eomenacanthus spp., Menacanthus spp., Trichodectes spp., Felicola spp., Damalinea spp., Bovicola spp;

from the order of the Diptera, for example, Chrysops spp., Tabanus spp., Musca spp., Hydrotaea spp., Muscina spp., Haematobosca spp., Haematobia spp., Stomoxys spp., Fannia spp., Glossina spp., Lucilia spp., Calliphora spp., Auchmeromyia spp., Cordylobia spp., Cochliomyia spp., Chrysomyia spp., Sarcophaga spp., Wohlfartia spp., Gasterophilus spp., Oesteromyia spp., Oedemagena spp., Hyporma spp., Oestrus spp., Rhinoestrus spp., Melophagus spp., Hippobosca spp.

From the order of the Siphonaptera, for example, Ctenocephalides spp., Echidnophaga spp., Ceratophyllus spp.

The action against fleas and ticks may be particularly emphasized.

Tick species which may be mentioned are: Ixodes spec., Rhipicephalus spec., Dermacentor spec., Haemaphysalis spec., Boophilus spec.; Hyalomma spec.

The domestic livestock and breeding animals include mammals such as, for example, cattle, horses, sheep, pigs, goats, camels, water buffalo, donkeys, rabbits, fallow deer, reindeer, fur bearers such as, for example, mink, chinchilla, racoon, and birds such as, for example, chickens, geese, turkeys and ducks.

Laboratory animals and experimental animals include mice, rats, guinea pigs, golden hamsters, dogs and cats.

The pets include dogs and cats.

The use in cats and dogs may be particularly emphasized.

The use may be prophylactic or else therapeutic.

To prepare the formulation according to the invention, suitable amounts of the desired components are mixed with each other, for example by using conventional stirred vessels or other suitable apparatuses.

The process may also carried out under a protective atmosphere or other methods of excluding oxygen, if the constituents require such a procedure.

The examples which follow are intended to illustrate the invention:

### EXAMPLE 1

A homogenous spot-on formulation (100 g) composed of

| | |
|---|---|
| 1.00 g | of pyriproxyfen (2-[1-methyl-2-(4-phenoxyphenoxy)ethoxy] |
| | pyridine (PPF) |
| 82.00 g | of diethylene glycol monoethyl ether |
| 15.00 g | of water |
| 2.00 g | of Abil Quat 3272 (1) |

Abil Quat 3272 is a commercially available 50% strength polydimethylsiloxane with a diquaternary ammonium group and a viscosity range of 1000+/−200 [mm².s−1] at 25° C. from Goldschmidt AG, D-4300 Essen.

### EXAMPLE 2

A homogenous spot-on formulation (100 g) composed of

| | |
|---|---|
| 1.00 g | of pyriproxyfen (2-[1-methyl-2-(4-phenoxyphenoxy)ethoxy] |
| | pyridine |
| 82.00 g | of dipropylene glycol monomethyl ether |
| 15.00 g | of water |
| 2.00 g | Abil Quat 3272 (1) |

Abil Quat 3272 is a commercially available 50% strength polydimethylsiloxane with a diquaternary ammonium group and a viscosity range of 1000+/−200[mm².s−1] at 25° C. from Goldschmidt AG, D-4300 Essen.

### EXAMPLE 3

A homogenous spot-on formulation (100 g) composed of

| | |
|---|---|
| 1.00 g | of pyriproxyfen (2-[1-methyl-2-(4-phenoxyphenoxy)ethoxy] |
| | pyridine |
| 82.00 g | of dipropylene glycol monomethyl ether |
| 15.00 g | of water |
| 2.00 g | of Abil Quat 3274 (2) |

(2) Abil Quat 3274 is a 50% strength polydimethylsiloxane solution with a diquaternary ammonium group and a viscosity range of 5000-15,000 [mm².s−1] at 25° C. from Goldschmidt AG, D-4300 Essen.

### EXAMPLE 4

A homogenous spot-on formulation (100 g) composed of

| | |
|---|---|
| 1.00 g | of pyriproxyfen (2-[1-methyl-2-(4-phenoxyphenoxy)ethoxy] |
| | pyridine |
| 82.00 g | of diethylene glycol monoethyl ether |
| 15.00 g | of water |
| 2.00 g | of Abil Quat 3274 (2) |

(2) Abil Quat 3274 is a 50% strength polydimethylsiloxane solution with a diquatemary ammonium group and a viscosity range of 5000-15,000 [mm².s−1] at 25° C. from Goldschmidt AG, D-4300 Essen.

### Comparative Example 1

A homogenous spot-on formulation (100 g) composed of

| | |
|---|---|
| 1.00 g | of pyriproxyfen (2-[1-methyl-2-(4-phenoxyphenoxy)ethoxy] |
| | pyridine |
| 99.00 g | of diethylene glycol monoethyl ether |

### Comparative Example 2

A homogenous spot-on formulation (100 g) composed of

| | |
|---|---|
| 1.00 g | of pyriproxyfen (2-[1-methyl-2-(4-phenoxyphenoxy)ethoxy] |
| | pyridine |
| 99.00 g | of dipropylene glycol monomethyl ether |

### Efficacy Studies on the Spot-on Formulations of Examples 1-4 on Fleas on Cats

The studies were carried out to prove the efficacy of the 1% strength pyriproxyfen solutions of the Examples for sterilizing fleas on cats.

In each case 20 flea-infested cats were divided into 2 groups of 10 cats each. One group received 0.1 ml/kg of pyriproxyfen spot-on 1% strength as spot-on on the neck, while the other group acted as untreated control. The cats were re-infested at one-week intervals, and the flea eggs shed were collected over a period of 24 hours. These eggs were incubated at 28° C. and a relative atmospheric humidity of 85% and observed over a period of 30 days for the development of fleas.

Moreover, in each case 20 cats were divided into 2 groups of 10 cats each. One group received 0.1 ml/kg of pyriproxyfen spot-on 1% strength as spot-on on the neck, while the other group acted as untreated control. Over a period of 4 weeks, the cats were placed at weekly intervals for in each case 6 hours on fleece blankets. Flea eggs were scattered on the blankets, and the eggs were incubated at 28° C. and a relative atmospheric humidity of 85% and observed over a period of 30 days for the development of fleas.

Over a period of 7 weeks, suppression of the development of adult fleas from eggs of the treatment group was at least 95% in comparison with the control group. The efficacy on day 56 p.appl. was 92%.

Over a period of 17 days, suppression of the development of adult fleas on blankets after contact with treated cats was 95% or more.

### Efficacy Studies on the Spot-on Formulations of Examples 1-4 on Fleas on Dogs

The studies were carried out to prove the efficacy of the 1% strength pyriproxyfen solutions of the Examples for sterilizing fleas on dogs.

In each case 20 flea-infested dogs were divided into 2 groups of 10 dogs each. One group received 0.04 ml/kg of pyriproxyfen spot-on 1% solutions of Examples 1-4 as spot-on on the neck, while the other group acted as untreated control. The dogs were re-infested at one-week intervals over a period of 11 weeks, and the flea eggs shed were collected over a period of 24 hours. These eggs were incubated at 28° C. and a relative atmospheric humidity of 85% and observed over a period of 30 days for the development of fleas.

Moreover, over a period of 4 weeks, the dogs were placed at weekly intervals for in each case 6 hours on fleece blankets. Flea eggs were scattered on the blankets, and the eggs were incubated at 28° C. and a relative atmospheric humidity of 85% and observed over a period of 30 days for the development of fleas.

Over a period of 10 weeks, suppression of the development of adult fleas from eggs of the treatment group was 100%.

Suppression of the development of adult fleas on blankets after contact with treated dogs was 100% over the entire test period of 24 days.

### Determination of the Long-Term Stability Behaviour

To this end, the conventional PP spot-on tubes with a mean wall thickness of approx. 350 μm were filled with in each case 0.4 ml of spot-on solution and stored for 6 months at 40° C. and a relative atmospheric humidity of 75%. The results of these studies are shown in Table 1.

TABLE 1
Long-term stability behaviour of the spot-on solutions

| Sample | | Water (%) | Water (%) | PPF (%) | PPF (%) |
|---|---|---|---|---|---|
| No. | Bulk product | (t / beginning) | (t / end) | (t / beginning) | (t / end) |
| 1 | Example 1 | 15 | 15 | 1.00 | 1.00 |
| 2 | Example 2 | 15 | 15 | 1.00 | 1.00 |
| 3 | Example 3 | 15 | 15 | 1.00 | 1.00 |
| 4 | Example 4 | 15 | 15 | 1.00 | 1.00 |
| 5 | Comparative Ex. 1 | 0 | 12 | 1.00 | 0.88 |
| 6 | Comparative Ex. 2 | 0 | 13 | 1.00 | 0.87 |

It can be seen from Table 1 that formulations 1-4 meet the stability requirements of the drug product regulations. Sadly, the formulations shown in Comparative Examples 1-2 are not storage-stable. In each case, >10% of the amounts of active compound in question are lost during storage.

The toxicological properties of the formulations in question are shown in Table 2.

TABLE 2
Toxicological properties of the spot-on solutions

| | Acute oral | Acute dermal | Skin irritation | Eye irritation in |
|---|---|---|---|---|
| Sample | LD50 in rats | LD50 in rats | in rabbits | rabbits |
| Bulk products of | >2500 | >4000 | non-irritant | non-irritant |
| Ex. 1-4 | (mg/kg b.w.) | (mg/kg b.w.) | | |
| Bulk products of | 2000 | >4000 | irritant | irritant |
| Comparative Ex. 1-2 | (mg/kg b.w.) | (mg/kg b.w.) | | |

It can be seen from the table that the new spot-on formulations exhibit an outstanding compatibility with the target animal and the environment and an outstanding user safety.

1. Use of polysiloxanes containing at least one quaternary ammonium group as formulation auxiliary in formulations of larvicidal and/or ovicidal active compounds.

2. Compositions containing
a) a larvicidal and/or ovicidal active compound and
b) a polysiloxane derivative with at least one quaternary ammonium group per molecule,
and, if appropriate, further auxiliaries and carriers.

3. Single-dose polypropylene polymer tubelets, characterized in that they are filled with a composition according to claim 2.
